(19) United States
(12) Patent Application Publication
(10) Pub. No.: US 2011/0097719 A1
(43) Pub. Date: Apr. 28, 2011

(54) Neprilysin Gene Polymorphism and Amyloid Beta Plaques in Traumatic Brain Injury

(75) Inventors: Douglas H. Smith, Boothwyn, PA (US); William Stewart, South Ayrshire (GB); Vicrtoria E. Johnson, Philadelphia, PA (US)

(73) Assignee: THE TRUSTEES OF THE UNIVERSITY OF PENNSYLVANIA, Philadelphia, PA (US)

(21) Appl. No.: 12/808,097

(22) PCT Filed: Dec. 10, 2008
(86) PCT No.: PCT/US2008/086265

§ 371 Date: Dec. 13, 2010

Related U.S. Application Data
(60) Provisional application No. 61/007,729, filed on Dec. 14, 2007.

(51) Int. Cl. C12Q 1/68

(52) U.S. Cl. 435/6

(57) ABSTRACT

The invention relates to methods of diagnosing risk of amyloid β deposition following traumatic brain injury. More particularly, the invention relates to the discovery of a specific single nucleotide polymorphism in the neprilysin gene that is linked to an increased risk of amyloid β deposition after traumatic brain injury

### BACKGROUND OF THE INVENTION

Traumatic brain injury (TBI) has the potential to trigger serious neurological sequelae, including progressive neurodegenerative disorders in ways not yet understood. In particular, TBI has been demonstrated to be an important epidemiological risk factor for the development of Alzheimer's Disease (AD). In addition, TBI and AD are linked by a common pathological finding. AD is characterized pathologically by deposits of amyloid plaques, neurofibrillary tangles, and neuronal death in the brain. Amyloid plaques are composed of amyloid β-peptide (Aβ peptide). The Aβ peptide is released from the amyloid β protein precursor (APP) by the action of two secretases, β and γ (reviewed in Haas, 2004, EMBO J. 23:483-488). Depending on the γ secretase, Aβ peptide can have 40 or 42 amino acid residues. While APP is a membrane-spanning protein, Aβ is a soluble peptide. Aβ peptide, however, is highly hydrophobic and readily self-aggregates, forming oligomers. Aggregation of Aβ oligomers results in fiber formation, and the fibers eventually precipitate and develop into the β amyloid plaques typical of Alzheimer's and other β amyloidogenic diseases.

Aβ levels within the brain are governed by a balance of its production and degradation. Neprilysin (NEP), also known as neutral endopeptidase, enkephalinase, CD 10 and common acute lymphoblastic leukemia antigen, can degrade amyloid β peptides. The nucleotide sequence of the neprilysin gene in man is exemplified in SEQ ID No. 1. The peptide sequence of the human neprilysin protein is exemplified in SEQ ID No. 2. The mRNA and protein levels of NEP are known to be reduced in the brains of Alzheimer's Disease patients. A GT-repeat polymorphism in the promoter region of the gene for neprilysin is associated with Alzheimer's Disease (Sakai et al., 2003, Dement. Geriatr. Cogn. Disord. 17:164-169). Specifically, the 19-repeat and 22-repeat alleles are overrepresented in AD patients. The shorter alleles of the GT-repeat polymorphism have been associated with cerebral amyloid angiopathy (CAA), a disorder characterized by cerebrovascular amyloid deposition, which causes intracerebral hemorrhages and other disorders (Yamada et al., 2003, J. Neur. Neurosurg. & Psych. 74:749-751).

AD-like Aβ plaques can be detected in about 30% of TBI cases within days of injury (see, for instance, Roberts et al., 1994, J. Neurol. Neurosurg. & Psych. 57:419-425). It is not known why some TBI patients, and not others, go on to develop Aβ plaques or why TBI is a significant risk factor for AD. Neither demographic nor clinical criteria that account for this apparent linkage in a sub-population of TBI patients have been identified. In addition, there is no method of identifying those TBI patients who are at risk for Aβ deposition. This invention meets that need.

### BRIEF SUMMARY OF THE INVENTION

The present invention is directed to a novel screening assay that allows the rapid and efficient identification of individuals at-risk for developing Aβ plaques in response to traumatic brain injury. As such, the present invention encompasses compositions and methods useful in the practice of the present invention.

In one embodiment, the invention is directed to a method of detecting a neprilysin gene polymorphism associated with amyloid β plaque development after traumatic brain injury (TBI) in an individual, where the method comprises obtaining a body sample from the individual, isolating genomic DNA from the sample, amplifying the neprilysin (NEP) gene from the genomic DNA; and detecting the number of GT tandem repeats present in the promoter region of the NEP gene. In the event that the individual expresses a NEP polymorphism having two alleles with a total of more than 41 GT repeats in the promoter of the NEP gene, the individual is at risk of developing amyloid β plaque after traumatic brain injury.

In another embodiment, the invention is directed to a method of detecting a neprilysin gene polymorphism associated with amyloid β plaque development after traumatic brain injury in an individual, where the method comprises obtaining a body sample from the individual, isolating genomic DNA from the sample, amplifying the neprilysin gene from the genomic DNA, and detecting the number of GT tandem repeats present in the promoter region of the NEP gene. In the event that the individual expresses a NEP polymorphism comprising a single allele encoding 22 GT repeats in the promoter of the NEP gene, the individual is at risk of developing amyloid β plaque after traumatic brain injury.

In yet another embodiment, the invention is directed to a method of detecting a neprilysin gene polymorphism associated with amyloid β plaque development after traumatic brain injury in an individual, where the method comprises obtaining a body sample from the individual, isolating genomic DNA from the sample, amplifying the neprilysin gene from the genomic DNA, and detecting the number of GT tandem repeats present in the promoter region of the NEP gene. In the event that the individual expresses a NEP polymorphism comprising a single allele encoding 20 GT repeats in the promoter of the NEP gene, the individual has a reduced risk of developing amyloid β plaque after traumatic brain injury.

In yet another embodiment, the invention is directed to a method of evaluating an individual's risk of developing an amyloid β plaque after traumatic brain injury, where the method comprises obtaining a body sample from the individual, isolating genomic DNA from the sample, amplifying the neprilysin gene from the genomic DNA, and detecting the number of GT tandem repeats present in the promoter region of the NEP gene. In the event that the individual expresses a NEP polymorphism having two alleles with a total of more than 41 GT repeats in the promoter of the NEP gene, the individual is at increased risk of developing amyloid β plaque after traumatic brain injury.

In yet another embodiment, the invention is directed to a method of evaluating an individual's risk of developing an amyloid β plaque after traumatic brain injury, where the method comprises obtaining a body sample from the individual, isolating genomic DNA from the sample, amplifying the neprilysin gene from the genomic DNA, and detecting the number of GT tandem repeats present in the promoter region of the NEP gene. In the event that the individual expresses a NEP polymorphism comprising a single allele encoding 22 GT repeats in the promoter of the NEP gene, the individual is at increased risk of developing amyloid β plaque after traumatic brain injury.

In yet another embodiment, the method is directed to a method of evaluating an individual's risk of developing an amyloid β plaque after traumatic brain injury, where the method comprises obtaining a body sample from the individual, isolating genomic DNA from the sample, amplifying the neprilysin gene from the genomic DNA, and detecting the number of GT tandem repeats present in the promoter region of the NEP gene. In the event that the individual expresses a NEP polymorphism comprising a single allele encoding 20 GT repeats in the promoter of the NEP gene, the individual is at a reduced risk of developing amyloid β plaque after traumatic brain injury.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is an example of the sequence of a GT repeat within the promoter region of the neprilysin gene (the neprilysin gene is exemplified in SEQ ID No. 1).

FIG. 2 is a graph depicting the number of each GT repeat allele present in members of the patient cohort that had amyloid plaques.

FIG. 3 is a graph depicting the number of each GT repeat allele present in members of the patient cohort who were in the control group, i.e. did not have amyloid plaques present.

FIG. 4 is a graph depicting the number of patients with amyloid plaques that had each genotype of interest.

FIG. 5 is a graph depicting the number of control patients without amyloid plaques that had each genotype of interest.

### DETAILED DESCRIPTION OF THE INVENTION

The present invention includes the development and implementation of a novel screening assay that allows the rapid and efficient identification of individuals at-risk for developing Aβ plaques in response to traumatic brain injury. As such, the present invention encompasses compositions and methods useful in the practice of the present invention.

### Definitions

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The phrase “biological sample”, as used herein, is intended to mean any sample comprising a cell, a tissue, or a bodily fluid obtained from an organism that can be assayed using the methods of the present invention to detect NEP gene polymorphisms. An example of such a biological sample includes a “body sample” obtained from a human patient. A “body sample” includes, but is not limited to, blood, lymph, urine, gynecological fluids, biopsies, amniotic fluid and smears. Samples that are liquid in nature are referred to herein as “bodily fluids.” Body samples may be obtained from a patient by a variety of techniques including, for example, by scraping or swabbing an area or by using a needle to aspirate bodily fluids. Methods for collecting various body samples are well known in the art.

As used herein, “elevated risk of developing Aβ plaques” refers to an individual with a genotype predictive of a greater likelihood of having or developing Aβ plaques in response to traumatic brain injury (TBI) as compared to another individual with a different genotype. Specifically, an individual with a single allele with 22 GT repeats in the NEP gene promoter (allele 22) is more likely to develop or have Aβ plaques in response to TBI than an individual who does not have allele 22. Similarly, an individual with two alleles that total more than 41 GT repeats is more likely to develop or have Aβ plaques in response to TBI than another individual having two alleles with a total number of GT repeats less than 41. Furthermore, the presence of an allele with 20 GT repeats is associated with reduced risk of having or developing Aβ plaques in response to TBI. In addition, the present invention contemplates distinguishing individuals at risk of developing Aβ plaques in response to TBI from individuals with AD.

An “allele,” as used herein, refers to one specific form of a genetic sequence (such as a gene) within a cell, an individual or within a population, the specific form differing from other forms of the same gene in the sequence of at least one, and frequently more than one, variant sites within the sequence of the gene. The sequence may or may not be within a gene. The sequences at these variant sites that differ between different alleles are termed “variances”, “polymorphisms”, or “mutations”. At each autosomal specific chromosomal location or “locus”, an individual possesses two alleles, one inherited from one parent and one from the other parent, for example one from the mother and one from the father. An individual is “heterozygous” at a locus if it has two different alleles at that locus. An individual is “homozygous” at a locus if it has two identical alleles at that locus.

“Polymorphism,” as used herein, refers to the occurrence of two or more genetically determined alternative sequences or alleles in a population. A polymorphic marker or site is the locus at which divergence occurs. Preferred markers have at least two alleles, each occurring at a frequency of preferably greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. A diallelic polymorphism has two forms. A triallelic polymorphism has three forms. A polymorphism between two nucleic acids can occur naturally, or be caused by exposure to or contact with chemicals, enzymes, or other agents, or exposure to agents that cause damage to nucleic acids, for example, ultraviolet radiation, mutagens or carcinogens.

The term “genotyping,” as used herein, refers to the determination of the genetic information an individual carries at one or more positions in the genome. For example, genotyping may comprise the determination of which allele or alleles an individual carries for a single polymorphism or the determination of which allele or alleles an individual carries for a plurality of polymorphisms. For example, a particular nucleotide in a genome may be an A in some individuals and a C in other individuals. Those individuals who have an A at the position have the A allele and those who have a C have the C allele. In a diploid organism the individual will have two copies of the sequence containing the polymorphic position so the individual may have an A allele and a C allele or alternatively two copies of the A allele or two copies of the C allele. Those individuals who have two copies of the C allele are homozygous for the C allele, those individuals who have two copies of the A allele are homozygous for the A allele, and those individuals who have one copy of each allele are heterozygous. The array may be designed to distinguish between each of these three possible outcomes. A polymorphic location may have two or more possible alleles and the array may be designed to distinguish between all possible combinations.

A “polynucleotide” means a single strand or parallel and anti-parallel strands of a nucleic acid. Thus, a polynucleotide may be either a single-stranded or a double-stranded nucleic acid. A polynucleotide is not defined by length and thus includes very large nucleic acids, as well as short ones, such as an oligonucleotide.

The term “nucleic acid” typically refers to large polynucleotides. In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”. Sequences on a DNA strand that are located 5′ to a reference point on the DNA are referred to as “upstream sequences”. Sequences on a DNA strand that are 3′ to a reference point on the DNA are referred to as “downstream sequences.”

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. Typical uses of primers include, but are not limited to, sequencing reactions and amplification reactions. A primer is typically single-stranded, but may be double-stranded. Primers are typically deoxyribonucleic acids, but a wide variety of synthetic and naturally-occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., detectable moieties, such as chromogenic, radioactive or fluorescent moieties, or moieties for isolation, e.g., biotin.

“Probe” refers to a polynucleotide that is capable of specifically hybridizing to a designated sequence of another polynucleotide. “Probe” as used herein encompasses oligonucleotide probes. A probe may or may not provide a point of initiation for synthesis of a complementary polynucleotide. A probe specifically hybridizes to a target complementary polynucleotide, but need not reflect the exact complementary sequence of the template. In such a case, specific hybridization of the probe to the target depends on the stringency of the hybridization conditions. For use in SNP detection, some probes are allele-specific, and hybridization conditions are selected such that the probe binds only to a specific SNP allele. Probes can be labeled with, e.g., detectable moieties, such as chromogenic, radioactive or fluorescent moieties, and used as detectable agents.

As used herein, “label” refers to a group covalently attached to a polynucleotide. The label may be attached anywhere on the polynucleotide but is preferably attached at one or both termini of the polynucleotide. The label is capable of conducting a function such as giving a signal for detection of the molecule by such means as fluorescence, chemiluminescence, and electrochemical luminescence. Alternatively, the label allows for separation or immobilization of the molecule by a specific or non-specific capture method (Andrus, 1995, In: PCR 2: A Practical Approach, McPherson et al. (Eds) Oxford University Press, Oxford, England, pp. 39-54). Labels include, but are not limited to, fluorescent dyes, such as fluorescein and rhodamine derivatives (U.S. Pat. Nos. 5,188,934 and 5,366,860), cyanine dyes, haptens, and energy-transfer dyes (Clegg, 1992, Methods Enzymol. 211:353-388; Cardullor et al., 1988, PNAS 85:8790-8794).

The term “target sequence”, “target nucleic acid” or “target” refers to a nucleic acid of interest. The target sequence may or may not be of biological significance. Typically, though not always, it is the significance of the target sequence that is being studied in a particular experiment. As non-limiting examples, target sequences may include regions of genomic DNA that are believed to contain one or more polymorphic sites, DNA encoding or believed to encode genes or portions of genes of known or unknown function, DNA encoding or believed to encode proteins or portions of proteins of known or unknown function, DNA encoding or believed to encode regulatory regions such as promoter sequences, splicing signals, polyadenylation signals, etc.

An “array” comprises a support, preferably solid, with nucleic acid probes attached to the support. Preferred arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854; 5,445,934; 5,744,305; 5,677,195; 5,800,992; 6,040,193 and 5,424,186, and Fodor et al., 1991, Science 251:767-777, each of which is incorporated by reference in its entirety for all purposes.

Arrays may generally be produced using a variety of techniques, such as mechanical synthesis methods or light directed synthesis methods that incorporate a combination of photolithographic methods and solid-phase synthesis methods. Techniques for the synthesis of these arrays using mechanical synthesis methods are described in, e.g., U.S. Pat. Nos. 5,384,261 and 6,040,193, which are incorporated herein by reference in their entirety for all purposes. Although a planar array surface is preferred, the array may be fabricated on a surface of virtually any shape or even a multiplicity of surfaces. Arrays may be nucleic acids on beads, gels, polymeric surfaces, fibers such as fiber optics, glass or any other appropriate substrate. (See U.S. Pat. Nos. 5,770,358; 5,789,162; 5,708,153; 6,040,193 and 5,800,992, which are hereby incorporated by reference in their entirety for all purposes.)

Arrays may be packaged in such a manner as to allow for diagnostic use or can be an all-inclusive device; e.g., U.S. Pat. Nos. 5,856,174 and 5,922,591 incorporated in their entirety by reference for all purposes. Preferred arrays are commercially available from Affymetrix (Santa Clara, Calif.) under the brand name GeneChip®, and are directed to a variety of purposes, including genotyping and gene expression monitoring for a variety of eukaryotic and prokaryotic species.

“Amplification” refers to any means by which a polynucleotide sequence is copied and thus expanded into a larger number of polynucleotide sequences, e.g., by reverse transcription, polymerase chain reaction or ligase chain reaction, among others.

“Hybridization probes,” as used herein, are oligonucleotides capable of binding in a base-specific manner to a complementary strand of nucleic acid. Such probes include peptide nucleic acids, as described in Nielsen et al., 1991, Science 254:1497-1500, and other nucleic acid analogs and nucleic acid mimetics. See U.S. patent application Ser. No. 08/630,427.

An “individual,” as used herein, is not limited to a human being, but may also include other organisms including but not limited to mammals, plants, fungi, bacteria or cells derived from any of the above.

As used herein, an “instructional material” includes a publication, a recording, a diagram, or any other medium of expression that can be used to communicate the usefulness of the kit for its designated use in practicing a method of the invention. The instructional material of the kit of the invention may, for example, be affixed to a container that contains the composition or be shipped together with a container that contains the composition. Alternatively, the instructional material may be shipped separately from the container with the intention that the instructional material and the composition be used cooperatively by the recipient.

### Description

According to the present invention, GT-repeat polymorphisms in the promoter region of the NEP gene are predictive of risk of having, or developing, amyloid β (Aβ) plaque deposition subsequent to a traumatic brain injury (TBI). Accordingly, the present invention provides methods of assessing risk of amyloid β plaque deposition in an individual in response to TBI. Kits useful in practicing embodiments of the inventive methods are also provided.

The methods and compositions of the present invention are applicable to any individual. The individual preferably is a human. The inventive methods and compositions are particularly indicated for individuals at risk of traumatic brain injury as well as TBI patients.

### I. Compositions.

### Alleles and Genotypes of the NEP Gene: GT Repeat Polymorphisms

The human NEP gene (exemplified in SEQ ID No. 1), located on chromosome 3q 25.1-q25.2, possesses four known types of dinucleotide repeats over ten repeat units long, including AC and GT repeats present in the upstream region of exon 1, a CA repeat in the intron 3, and a TG repeat in intron 22.

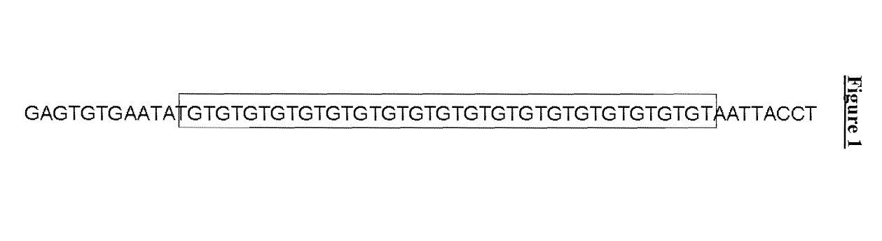

The GT dinucleotide polymorphisms upstream of exon 1 are located in the promoter region of the NEP gene (Coming et al., 1999, Neuroreport 10:2283-2285; Comings et al., 2000, Psych. Genetics, 10:91-94, encompassed in their entirety herein). These polymorphisms comprise a short tandem repeat consisting of GT repeats (see, for example, FIG. 1). An individual genotype for a GT polymorphism comprises two alleles and may be either homozygous or heterozygous. Each allele is defined by the number of GT repeats found for that polymorphism and is named accordingly. Thus, individual alleles comprising 19, 20, 21, 22, and 23 GT repeats within the NEP promoter are named allele 19, allele 20, allele 21, allele 22, and allele 23, respectively. The number of repeats varies from about 19 to about 23 tandem GT repeats per allele.

The genotype for an individual is defined by the total number of GT repeats contributed by each allele present in an individual, and may comprise any of the following combinations of alleles: 19/19, 19/20; 19/21; 19/22; 19/23, 20/20; 20/21; 20/22; 20/23; 21/20, 21/21; 21/22; and 21/23. Accordingly, the range for an individual polymorphism is from about 38 to about 46 total GT repeats.

One embodiment of the present invention includes compositions and methods useful in the identification of an individual with elevated risk of developing Aβ plaques in response to TBI. In one embodiment of the present invention, an individual has a genotype for a GT repeat polymorphism of the NEP gene comprising allele 22 indicating that said individual has an elevated risk of developing Aβ plaques in response to TBI. In another embodiment of the present invention, an individual has a genotype for a GT repeat polymorphism of the NEP gene comprising two alleles with a total number of GT repeats greater than 41 indicating that said individual has an elevated risk of developing Aβ plaques in response to TBI. In yet another embodiment of the present invention, an individual has a genotype for a GT repeat polymorphism of the NEP gene comprising two alleles with a total GT repeat less than 41 indicating that said individual has a reduced risk of developing Aβ plaques in response to TBI. In still another embodiment of the present invention, an individual has a genotype for a GT repeat polymorphism of the NEP gene comprising allele 20 indicating that said individual has a reduced risk of developing Aβ plaques in response to TBI. Alleles 19 and 22, thus a genotype with a total of exactly 41 GT repeats, are overrepresented in patients with late onset AD. Accordingly, another embodiment of the present invention distinguishes an individual with AD from an individual at risk of developing Aβ plaques in response to TBI based on the total number of GT repeats present in the promoter of the NEP gene.

### Nucleic Acids: Primers

The present invention encompasses isolated nucleic acids useful in the practice of the methods of the invention. Specifically, the present invention encompasses primers useful in the amplification of dinucleotide GT polymorphisms located in the promoter region of the NEP gene. Each primer should be sufficiently long to initiate or prime the synthesis of extension DNA products in the presence of an appropriate polymerase and other reagents. Appropriate primer length is dependent on many factors, as is well known; typically, in the practice of applicant's method, a primer will be used that contains 15-30 nucleotide residues. Short primer molecules generally require lower reaction temperatures to form and to maintain the primer-template complexes that support the chain extension reaction.

The primers used need to be substantially complementary to the nucleic acid containing the selected sequences to be amplified, i.e, the primers must bind to, i.e. hybridize with, nucleic acid containing the selected sequence (or its complement). The primer sequence need not be entirely an exact complement of the template; for example, a non-complementary nucleotide fragment or other moiety may be attached to the 5′ end of a primer, with the remainder of the primer sequence being complementary to the selected nucleic acid sequence. Primers that are fully complementary to the selected nucleic acid sequence are preferred and typically used.

In a preferred embodiment of the present invention to diagnose for a polymorphism of the GT tandem repeat present in the promoter region of the NEP gene, a pair of primers is used. One of the primers, i.e., the forward primer, is complementary to a sequence which is near, abuts and/or includes the 3′ end of the antisense of the selected DNA region. The other primer, i.e., the reverse primer, contains the complement of the sequence which is near, abuts or and/or includes the complement of the sequence at the 3′ end of the selected DNA region.

Generally, primers will be between about 15 and 30 nucleotides in length and preferably between about 18 and 27 nucleotides in length. They are preferably chosen to hybridize to a unique DNA sequence in the genome so as to maximize the desired location hybridization that will occur.

In one embodiment of the invention, the forward primers of the pair of primers that are used preferably have an anchoring moiety covalently linked to the 5′ end of each primer. The reverse primers are derivatized with phosphate at the 5′ ends. Generally, any anchoring moiety can be used that will serve to couple the oligonucleotide to a solid surface or solid phase.

As is well known in this art, various solid phase material can be used; for example, the solid support material can be selected from any of a wide variety of materials that are commonly used, such as those that are commercially available from Amersham Biosciences, BioRad, and Sigma. It can be in the form of particles, plates, matrices, fibers or the like, and it may be made of silica, cellulose, agarose beads, controlled-pore glass, polymeric beads, gel beads, or magnetic beads. Magnetic beads are preferred because the use of such facilitates their subsequent separation from the supernatant by the straightforward application of a magnetic field. Such can be done using flow chambers or by simply pipetting. Such magnetic beads, for example those sold as Dynal beads or those sold by Advanced Magnetics, can be used to separate the amplified DNA from the remainder of the biological sample and the PCR material and reaction products by washing. This same property is also used to advantage in separating decoupled target material, at a later stage in the assay procedure. Although the particles in bead form are preferred for facility and handling, other shaped particles or substrates might alternatively be employed. Such commercially available magnetic beads are generally small non-porous spheres that are coated with a layer of magnetite to provide the desired magnetic properties, and then with an exterior coating. Magnetic beads that are commercially available for these purposes are produced in various ways; often paramagnetic metals, such as metal oxides, are encapsulated with a suitable coating material, such as a polymer or a silicate, to produce coated beads that are about 1 μm-100 μm in diameter.

Anchoring moieties and coupling agents that are complementary and bind to each other are used as a linkage to attach the amplified DNA to such solid support. Many varieties of binding pairs are well known in the art and may be suitably employed. The anchoring moiety may join directly to the solid phase or, more usually, to a complementary coupling agent carried by the solid phase. A preferred binding system employs avidin or streptavidin and biotin. Streptavidin, for example, is covalently attached to the exterior surface of the solid support, e.g., the magnetic beads, and it, in turn, binds strongly to biotinylated DNA. Such magnetic beads suitable for applications of interest are commercially available from a number of vendors. Beads that have streptavidin bound to the surface of the beads, having a nominal size of about 1 micron in diameter, are sold by Active Motif of Carlsbad, Calif. Other binding pairs, e.g. antibody-antigen and the like, may alternatively be used as such an intermediate linkage.

The length of nucleic acid along the chromosome in question that will be amplified will of course be determined by the length of the GT tandem repeat region present in the NEP promoter as it is this variation in length toward which the method of the invention is directed.

### Nucleic Acids: Target Sequences

The target sequence or target nucleic acid may be a portion of a gene, a regulatory sequence, genomic DNA, cDNA, and RNA (including mRNA and rRNA). Genomic DNA samples are usually amplified before being brought into contact with a probe. Genomic DNA can be obtained from any biological sample, including, by way of non-limiting example, tissue source or circulating cells (other than pure red blood cells). For example, convenient sources of genomic DNA include whole blood, semen, saliva, tears, urine, fecal material, sweat, buccal cells, skin and hair. Amplification of genomic DNA containing a polymorphic site generates a single species of target nucleic acid if the individual from which the sample was obtained is homozygous at the polymorphic site, or two species of target molecules if the individual is heterozygous. RNA samples also are often subject to amplification. In this case, amplification is typically preceded by reverse transcription. Amplification of all expressed mRNA can be performed as described in, for example, PCT Publication Nos. WO96/14839 and WO97/01603, which are hereby incorporated by reference in their entirety. Amplification of an RNA sample from a diploid sample can generate two species of target molecules if the individual providing the sample is heterozygous at a polymorphic site occurring within the expressed RNA, or possibly more if the species of the RNA is subjected to alternative splicing. Amplification generally can be performed using the polymerase chain reaction (PCR) methods known in the art. Nucleic acids in a target sample can be labeled in the course of amplification by inclusion of one or more labeled nucleotides in the amplification mixture. Labels also can be attached to amplification products after amplification (e.g., by end-labeling). The amplification product can be RNA or DNA, depending on the enzyme and substrates used in the amplification reaction.

In one embodiment of the invention, the target nucleic acid is the human NEP gene. In one aspect of the present invention, the target nucleic acid comprises a tandem repeat present on a single allele, said repeat comprising a variable number of GT dinucleotide repeats in the promoter region of the NEP gene, as described elsewhere herein. These GT repeats are represented by various alleles and may range in length from about 19 GT repeats to about 23 GT repeats per allele. In another aspect of the present invention, the target nucleic acid comprises a tandem repeat present on more than one alleles of the NEP gene.

The genotype of an individual polymorphism comprises the sum of at least two alleles and may be heterozygous (i.e. comprising identical alleles) or heterozygous (i.e. comprising different alleles). In one aspect of the invention, the target nucleic acid comprises a single allele of the human NEP gene. In another aspect of the invention, the target nucleic acid comprises more than one allele of the human NEP gene. In another aspect of the invention, the total number of GT repeats present on all alleles is assessed using the methods of the invention, wherein the total number of GT dinucleotide repeats ranges from about 38 to about 46 GT repeats. In one aspect of the invention wherein the marker is a GT tandem repeat, the number of tandem repeats in a target nucleic acid sequence may be determined by parallel interrogation as elaborated herein.

### Nucleic Acids: Synthesis

An isolated nucleic acid of the present invention can be produced using conventional nucleic acid synthesis or by recombinant nucleic acid methods known in the art (2001, Molecular Cloning: A Laboratory Manual, Cold Spring Harbor Laboratory Press, New York) and Ausubel et al. (2001, Current Protocols in Molecular Biology, Green & Wiley, New York).

### Tags

In one embodiment of the invention, an isolated nucleic acid of the invention comprises a covalently linked tag. By way of a non-limiting example, an isolated nucleic acid of the present invention may comprise a primer, an oligonucleotide, and a target sequence. That is, the invention encompasses a chimeric nucleic acid wherein the isolated nucleic acid sequence comprises a tag molecule. Such tag molecules are well known in the art and include, for instance, a ULS reagent that reacts with the N-7 position of guanine residues, an amine-modified nucleotide, a 5-(3-aminoallyl)-dUTP, an amine-reactive succinimidyl ester moiety, a biotin molecule, ³³P, ³²P, fluorescent labels such as fluorescein (FITC), 5,6-carboxymethyl fluorescein, Texas Red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7.

However, the invention should in no way be construed to be limited to the nucleic acids encoding the above-listed tags. Rather, any tag that may function in a manner substantially similar to these tag polypeptides should be construed to be included in the present invention.

The isolated nucleic acid comprising a tag can be used to localize an isolated nucleic acid, for example, within a cell, a tissue, and/or a whole organism (e.g., a mammalian embryo), detect an isolated nucleic acid, for example, in a cell, and to study the role(s) of an isolated nucleic acid in a cell. Further, addition of a tag facilitates isolation and purification of the isolated nucleic acid.

### II. Methods.

The compositions and methods of the present invention may be used to identify a polymorphism of the NEP gene present in an individual. In one embodiment, the present invention may be used to identify a specific allele present in an individual wherein that allele encodes a varying GT tandem repeat in the promoter region of the NEP gene. In another embodiment, the present invention may be used to determine the genotype of an individual for both alleles encoding a GT tandem repeat in the promoter of the NEP gene. In another embodiment, the present invention may be used to determine the total number of GT repeats present in the promoter region of the NEP gene of an individual. In still another embodiment, the present invention may be used to determine an individual's risk of developing Aβ plaques in response to traumatic head injury.

### Methods of Identifying NEP Polymorphisms

A number of methods are available for analysis of polymorphisms. Assays for detection of polymorphisms or mutations fall into several categories, including but not limited to direct sequencing assays, fragment polymorphism assays, hybridization assays, and computer based data analysis. Protocols and commercially available kits or services for performing multiple variations of these assays are available. In some embodiments, assays are performed in combination or in hybrid (e.g., different reagents or technologies from several assays are combined to yield one assay). The following assays are useful in the present invention, and are described in relationship to detection of the various GT repeat alleles found in the NEP promoter. However, the present invention is not limited to detection of the allele or alleles of the NEP gene described herein. In fact, detection of at least one allele of the human NEP gene associated with increased Aβ deposition in response to traumatic brain injury are within the scope of the present invention.

### 1. Direct Sequencing Assays

In some embodiments of the present invention, polymorphisms are detected using a direct sequencing technique. In these assays, DNA samples are first isolated from a subject using any suitable method. In some embodiments, the region of interest is cloned into a suitable vector and amplified by growth in a host cell (e.g., a bacterium). In other embodiments, DNA in the region of interest is amplified using PCR.

Following amplification, DNA in the region of interest (e.g., the region containing the polymorphism of interest) is sequenced using any suitable method, including but not limited to manual sequencing using radioactive marker nucleotides, or automated sequencing. The results of the sequencing are displayed using any suitable method. The sequence is examined and the presence or absence of a given polymorphism is determined.

### 2. PCR Assays

In some embodiments of the present invention, polymorphisms are detected using a PCR-based assay. In some embodiments, the PCR assay comprises the use of oligonucleotide primers to amplify a fragment containing the repeat polymorphism of interest. The presence of an additional repeat in the gene results in the generation of a longer PCR fragment that can be detected by gel electrophoresis. For instance, by use of the method described in Example 2, the allele encoding a GT tandem repeat in the NEP gene is detected by the appearance of a shorter (e.g., 19 GT repeat) or longer (e.g., 23 GT repeat) repeat in the gene results.

Amplification of a target polynucleotide sequence may be carried out by any method known to the skilled artisan. See, for instance, Kwoh et al. (1990, Am. Biotechnol. Lab. 8:14-25) and Hagen-Mann, et al., (1995, Exp. Clin. Endocrinol. Diabetes 103:150-155). Amplification methods include, but are not limited to, polymerase chain reaction (“PCR”) including RT-PCR, strand displacement amplification (Walker et al., 1992, PNAS, 89:392-396; Walker et al., 1992, Nucleic Acids Res. 20:1691-1696), strand displacement amplification using Phi29 DNA polymerase (U.S. Pat. No. 5,001,050), transcription-based amplification (Kwoh et al., 1989, PNAS 86:1173-1177), self-sustained sequence replication (“3SR”) (Guatelli et al., 1990, PNAS 87:1874-1878; Mueller et al., 1997, Histochem. Cell Biol. 108:431-437), the Q.beta. replicase system (Lizardi et al., 1988, BioTechnology 6:1197-1202; Cahill et al., 1991, Clin. Chem. 37:1482-1485), nucleic acid sequence-based amplification (“NASBA”) (Lewis, 1992, Gen. Eng. News 12 (9):1), the repair chain reaction (“RCR”) (Lewis, 1992, supra), and boomerang DNA amplification (or “BDA”) (Lewis, 1992, supra). PCR is the preferred method of amplifying the target polynucleotide sequence.

PCR may be carried out in accordance with known techniques. See, e.g., Bartlett et al., eds., 2003, PCR Protocols Second Edition, Humana Press, Totowa, N.J. and U.S. Pat. Nos. 4,683,195; 4,683,202; 4,800,159 and 4,965,188. In general, PCR involves, first, treating a nucleic acid sample (e.g., in the presence of a heat stable DNA polymerase) with a pair of amplification primers. One primer of the pair hybridizes to one strand of a target polynucleotide sequence. The second primer of the pair hybridizes to the other, complementary strand of the target polynucleotide sequence. The primers are hybridized to their target polynucleotide sequence strands under conditions such that an extension product of each primer is synthesized which is complementary to each nucleic acid strand. The extension product synthesized from each primer, when it is separated from its complement, can serve as a template for synthesis of the extension product of the other primer. After primer extension, the sample is treated to denaturing conditions to separate the primer extension products from their templates. These steps are cyclically repeated until the desired degree of amplification is obtained.

The amplified target polynucleotide may be used in one of the detection assays described elsewhere herein to identify the GT-repeat polymorphism present in the amplified target polynucleotide sequence.

### 3. Fragment Length Polymorphism Assays

In some embodiments of the present invention, polymorphisms are detected using a fragment length polymorphism assay. In a fragment length polymorphism assay, a unique DNA banding pattern based on cleaving the DNA at a series of positions is generated using an enzyme (e.g., a restriction endonuclease). DNA fragments from a sample containing a polymorphism will have a different banding pattern than wild type.

In one embodiment of the present invention, fragment sizing analysis is carried out using the Beckman Coulter CEQ 8000 genetic analysis system, a method well-known in the art for microsatellite polymorphism determination.

a. RFLP Assay

In some embodiments of the present invention, polymorphisms are detected using a restriction fragment length polymorphism assay (RFLP). The region of interest is first isolated using PCR. The PCR products are then cleaved with restriction enzymes known to give a unique length fragment for a given polymorphism. The restriction-enzyme digested PCR products are separated by agarose gel electrophoresis and visualized by ethidium bromide staining. The length of the fragments is compared to molecular weight markers and fragments generated from control subjects not expressing Aβ plaques.

b. CFLP Assay

In other embodiments, polymorphisms are detected using a CLEAVASE fragment length polymorphism assay (CFLP; Third Wave Technologies, Madison, Wis.; see e.g., U.S. Pat. No. 5,888,780). This assay is based on the observation that, when single strands of DNA fold on themselves, they assume higher order structures that are highly individual to the precise sequence of the DNA molecule. These secondary structures involve partially duplexed regions of DNA such that single stranded regions are juxtaposed with double stranded DNA hairpins. The CLEAVASE I enzyme, is a structure-specific, thermostable nuclease that recognizes and cleaves the junctions between these single-stranded and double-stranded regions.

The region of interest is first isolated, for example, using PCR. Then, DNA strands are separated by heating. Next, the reactions are cooled to allow intrastrand secondary structure to form. The PCR products are then treated with the CLEAVASE I enzyme to generate a series of fragments that are unique to a given polymorphism. The CLEAVASE enzyme treated PCR products are separated and detected (e.g., by agarose gel electrophoresis) and visualized (e.g., by ethidium bromide staining). The length of the fragments is compared to molecular weight markers and fragments generated from wild-type and mutant controls.

### 4. Hybridization Assays

In other embodiments of the present invention, polymorphisms are detected by hybridization assay. In a hybridization assay, the presence or absence of a given polymorphism or mutation is determined based on the ability of the DNA from the sample to hybridize to a complementary DNA molecule (e.g., an oligonucleotide probe). A variety of hybridization assays using a variety of technologies for hybridization and detection are available. A description of a selection of assays is provided below.

In a preferred embodiment, the hybridized nucleic acids are detected by detecting one or more labels attached to the sample nucleic acids. The labels may be incorporated by any of a number of means well known to those of skill in the art. In one embodiment, the label is simultaneously incorporated during the amplification step in the preparation of the sample nucleic acids. Thus, for example, polymerase chain reaction (PCR) with labeled primers or labeled nucleotides will provide a labeled amplification product. In another embodiment, transcription amplification using a labeled nucleotide (e.g. fluorescein-labeled UTP and/or CTP) incorporates a label into the transcribed nucleic acids.

Alternatively, a label may be added directly to the original nucleic acid sample (e.g., mRNA, polyA mRNA, cDNA, etc.) or to the amplification product after the amplification is completed. Means of attaching labels to nucleic acids are well known to those of skill in the art and include, for example, nick translation or end-labeling (e.g. with a labeled RNA) by kinasing the nucleic acid and subsequent attachment (ligation) of a nucleic acid linker joining the sample nucleic acid to a label (e.g., a fluorophore). In another embodiment label is added to the end of fragments using terminal deoxytransferase (TdT).

Detectable labels suitable for use in the present invention include any composition detectable by spectroscopic, photochemical, biochemical, immunochemical, electrical, optical or chemical means. Useful labels in the present invention include, but are not limited to: biotin for staining with labeled streptavidin conjugate; anti-biotin antibodies; magnetic beads (e.g., Dynabeads™); fluorescent dyes (e.g., fluorescein, Texas Red, rhodamine, green fluorescent protein, and the like); radiolabels (e.g., ³H, ¹²⁵I, ³⁵S, ¹⁴C, or ³²P); phosphorescent labels; enzymes (e.g., horse radish peroxidase, alkaline phosphatase and others commonly used in an ELISA); and calorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149 and 4,366,241, each of which is hereby incorporated by reference in its entirety for all purposes.

Means of detecting such labels are well known to those of skill in the art. Thus, for example, radiolabels may be detected using photographic film or scintillation counters; fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label.

The label may be added to the target nucleic acid(s) prior to, or after the hybridization. So-called “direct labels” are detectable labels that are directly attached to or incorporated into the target nucleic acid prior to hybridization. In contrast, so-called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids. See Tijssen, 1993, Laboratory Techniques in Biochemistry and Molecular Biology, Vol. 24: Hybridization with Nucleic Acid Probes, which is hereby incorporated by reference in its entirety for all purposes.

a. Direct Detection of Hybridization

In some embodiments, hybridization of a probe to the sequence of interest (e.g., polymorphism) is detected directly by visualizing a bound probe (e.g., a Northern or Southern assay; See e.g., Ausabel et al. (Eds.), 1991, Current Protocols in Molecular Biology, John Wiley & Sons, NY. In these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., agarose gel electrophoresis) and transferred to a membrane. A labeled (e.g., by incorporating a radionucleotide) probe or probes specific for the mutation being detected is allowed to contact the membrane under a condition of low, medium, or high stringency conditions. Unbound probe is removed and the presence of binding is detected by visualizing the labeled probe.

b. Detection of Hybridization Using “DNA Chip” Assays

In some embodiments of the present invention, polymorphisms and/or differences in levels of gene expression (e.g., mRNA) are detected using a DNA chip hybridization assay. In this assay, a series of oligonucleotide probes are affixed to a solid support. The oligonucleotide probes are designed to be unique to a given polymorphism. The DNA sample of interest is contacted with the DNA “chip” and hybridization is detected.

In some embodiments, the DNA chip assay is a GeneChip (Affymetrix, Santa Clara, Calif.; see e.g., U.S. Pat. No. 6,045,996) assay. The GeneChip technology uses miniaturized, high-density arrays of oligonucleotide probes affixed to a “chip”. Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In other embodiments, a DNA microchip containing electronically captured probes (Nanogen, San Diego, Calif.) is utilized (see e.g., U.S. Pat. No. 6,068,818). Through the use of microelectronics, Nanogen's technology enables the active movement and concentration of charged molecules to and from designated test sites on its semiconductor microchip. DNA capture probes unique to a given polymorphism or mutation are electronically placed at, or “addressed” to, specific sites on the microchip. Since DNA has a strong negative charge, it can be electronically moved to an area of positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.

A test sample is then analyzed for the presence of target DNA molecules by determining which of the DNA capture probes hybridize, with complementary DNA in the test sample (e.g., a PCR amplified gene of interest). An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site, the polarity or charge of the site is reversed to negative, thereby forcing any unbound or nonspecifically bound DNA back into solution away from the capture probes. A laser-based fluorescence scanner is used to detect binding.

In still further embodiments, an array technology based upon the segregation of fluids on a flat surface (chip) by differences in surface tension (ProtoGene, Palo Alto, Calif.) is utilized (see e.g., U.S. Pat. No. 6,001,311). Protogene's technology is based on the fact that fluids can be segregated on a flat surface by differences in surface tension that have been imparted by chemical coatings. Once so segregated, oligonucleotide probes are synthesized directly on the chip by ink jet printing of reagents. The array with its reaction sites defined by surface tension is mounted on a X/Y translation stage under a set of four piezoelectric nozzles, one for each of the four standard DNA bases. The translation stage moves along each of the rows of the array, and the appropriate reagent is delivered to each of the reaction site. For example, the A amidite is delivered only to the sites where amidite A is to be coupled during that synthesis step and so on. Common reagents and washes are delivered by flooding the entire surface followed by removal by spinning.

DNA probes unique for the polymorphism of interest are affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent group).

In yet other embodiments, a “bead array” is used for the detection of polymorphisms (Illumina, San Diego, Calif.; see e.g., PCT Publications WO99/67641 and WO00/39587, each of which is herein incorporated by reference). Illumina uses a BEAD ARRAY technology that combines fiber optic bundles and beads that self-assemble into an array. Each fiber optic bundle contains thousands to millions of individual fibers depending on the diameter of the bundle. The beads are coated with an oligonucleotide specific for the detection of a given polymorphism or mutation. Batches of beads are combined to form a pool specific to the array. To perform an assay, the BEAD ARRAY is contacted with a prepared subject sample (e.g., DNA). Hybridization is detected using any suitable method.

c. Enzymatic Detection of Hybridization

In some embodiments of the present invention, genomic profiles are generated using an assay that detects hybridization by enzymatic cleavage of specific structures (INVADER assay, Third Wave Technologies; see e.g., U.S. Pat. No. 6,001,567). The INVADER assay detects specific DNA and RNA sequences by using structure-specific enzymes to cleave a complex formed by the hybridization of overlapping oligonucleotide probes. Elevated temperature and an excess of one of the probes enable multiple probes to be cleaved for each target sequence present without temperature cycling. These cleaved probes then direct cleavage of a second labeled probe. The secondary probe oligonucleotide can be 5′-end labeled with fluorescein that is quenched by an internal dye. Upon cleavage, the dequenched fluorescein labeled product may be detected using a standard fluorescence plate reader.

The INVADER assay detects specific mutations and polymorphisms in unamplified genomic DNA. The isolated DNA sample is contacted with the first probe specific either for a polymorphism/mutation or wild type sequence and allowed to hybridize. Then a secondary probe, specific to the first probe, and containing the fluorescein label, is hybridized and the enzyme is added. Binding is detected using a fluorescent plate reader and comparing the signal of the test sample to known positive and negative controls.

In some embodiments, hybridization of a bound probe is detected using a TaqMan assay (PE Biosystems, Foster City, Calif.; see e.g., U.S. Pat. No. 5,962,233). The assay is performed during a PCR reaction. The TaqMan assay exploits the 5′-3′ exonuclease activity of the AMPLITAQ GOLD DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

### 5. Mass Spectroscopy Assay

In some embodiments, a MassARRAY system (Sequenom, San Diego, Calif.) is used to detect polymorphisms (see e.g., U.S. Pat. No. 6,043,031). DNA is isolated from blood samples using standard procedures. Next, specific DNA regions containing the polymorphism of interest are amplified by PCR. The amplified fragments are then attached by one strand to a solid surface and the non-immobilized strands are removed by standard denaturation and washing. The remaining immobilized single strand then serves as a template for automated enzymatic reactions that produce genotype specific diagnostic products.

Very small quantities of the enzymatic products, typically five to ten nanoliters, are then transferred to a SpectroCHIP array for subsequent automated analysis with the SpectroREADER mass spectrometer. Each spot is preloaded with light absorbing crystals that form a matrix with the dispensed diagnostic product. The MassARRAY system uses MALDI-TOF (Matrix Assisted Laser Desorption Ionization-Time of Flight) mass spectrometry. In a process known as desorption, the matrix is hit with a pulse from a laser beam. Energy from the laser beam is transferred to the matrix and it is vaporized resulting in a small amount of the diagnostic product being expelled into a flight tube. As the diagnostic product is charged when an electrical field pulse is subsequently applied to the tube they are launched down the flight tube towards a detector. The time between application of the electrical field pulse and collision of the diagnostic product with the detector is referred to as the time of flight. This is a very precise measure of the product's molecular weight, as a molecule's mass correlates directly with time of flight with smaller molecules flying faster than larger molecules. The entire assay is completed in less than 0.0001 second, enabling samples to be analyzed in a total of 3-5 second including repetitive data collection. The SpectroTYPER software then calculates, records, compares and reports, the genotypes at the rate of three seconds per sample.

### III. Kits

The invention encompasses various kits relating to compositions and methods used to identify a polymorphism of the NEP gene present in an individual, preferably a human. In one embodiment, the kit may be used to identify a specific allele present in an individual wherein that allele encodes a GT tandem repeat polymorphism in the promoter region of the NEP gene. In another embodiment, the kit may be used to determine the genotype of an individual for both alleles encoding a GT tandem repeat polymorphism in the promoter of the NEP gene. In another embodiment, the kit may be used to determine the total number of GT repeats present in a polymorphism of the NEP gene of an individual. In still another embodiment, the kit may be used to determine an individual's risk of developing Aβ plaques in response to traumatic head injury.

The kit comprises an isolated nucleic acid, preferably a primer, a set of primers, or an array of primers, as described elsewhere herein. The primers may be fixed to a solid substrate, as described elsewhere herein. The kit may further comprise a control target nucleic acid and primers. The isolated nucleic acids of the kit may also comprise a molecular label or tag. In additional embodiments, the kits of the present invention comprise various reagents necessary to practice the methods of the invention, as disclosed herein. The kit further comprises instructional material for the use thereof to be used in accordance with the teachings provided herein.

### IV. Methods of Use

The methods of the presently claimed invention can be used for a wide variety of applications including, for example, linkage and association studies, genotyping clinical populations, correlation of genotype information to phenotype information, identification and counseling of at-risk populations. Any analysis of genomic DNA may be benefited by a reproducible method of polymorphism analysis.

In a preferred embodiment, the methods of the presently claimed invention are used to genotype individuals, populations or samples. For example, any of the procedures described above, alone or in combination, could be used to interrogate samples obtained from a large number of individuals. Arrays may be designed and manufactured on a large scale basis to interrogate those fragments with probes comprising sequences that encompass the GT tandem repeat region of the NEP promoter. Thereafter, a sample from one or more individuals would be obtained and prepared using the same techniques which were used to prepare the selection probes or to design the array. Each sample can then be hybridized to an array and the hybridization pattern can be analyzed to determine the genotype of each individual or a population of individuals. Methods of use for polymorphisms and SNP discovery can be found in, for example, U.S. Pat. No. 6,361,947, which is herein incorporated by reference in its entirety for all purposes.

Correlation of Polymorphisms with Phenotypic Traits

Most human sequence variation is attributable to or correlated with single nucleotide polymorphisms (SNPs), with the rest attributable to insertions or deletions of one or more bases, repeat length polymorphisms and rearrangements. Human diversity is limited not only by the number of SNPs occurring in the genome but further by the observation that specific combinations of alleles are found at closely linked sites, generating haplotypes. For a description of haplotypes see, for example, Gabriel et al., 2002, Science 296:2225-9; Daly et al., 2001, Nat. Genet. 29:229-32; and Rioux et al., 2001, Nat. Genet. 29: 223-8, each of which is incorporated herein by reference in its entirety.

Correlation of individual polymorphisms or groups of polymorphisms with phenotypic characteristics is a valuable tool in the effort to identify DNA variation that contributes to population variation in phenotypic traits. Phenotypic traits include, for example, physical characteristics, risk for disease, and response to the environment. Polymorphisms that correlate with disease are particularly interesting because they represent mechanisms to accurately diagnose disease and targets for drug treatment, or identify and counsel individuals at-risk for developing disease. Hundreds of human diseases have already been correlated with individual polymorphisms but there are many diseases that are known to have an, as yet unidentified, genetic component and many diseases for which a component is or may be genetic.

Many diseases may correlate with multiple genetic changes making identification of the polymorphisms associated with a given disease more difficult. One approach to overcome this difficulty is to systematically explore the limited set of common gene variants for association with disease.

To identify correlation between one or more alleles and one or more phenotypic traits, individuals are tested for the presence or absence of polymorphic markers or marker sets and for the phenotypic trait or traits of interest. The presence or absence of a set of polymorphisms is compared for individuals who exhibit a particular trait and individuals who exhibit lack of the particular trait to determine if the presence or absence of a particular allele is associated with the trait of interest. For example, it might be found that the presence of allele Al at polymorphism A correlates with heart disease. As an example of a correlation between a phenotypic trait and more than one polymorphism, it might be found that allele Al at polymorphism A and allele B1 at polymorphism B correlate with a phenotypic trait of interest.

The present invention discloses a method of identifying individuals with a greater than normal risk of developing serious neuropathology secondary to traumatic head injury based upon alleles encoding GT tandem repeats in the promoter region of the NEP gene as well as the total number of GT tandem repeats present in the NEP gene. Accordingly, the present invention allows individuals engaged in behaviors that carry a heightened risk of head trauma, such as contact sports, the military, and motorcycle use, to be identified and counseled as to methods of reducing their risk of long-term neurological damage.

### Diagnosis of Disease and Predisposition to Disease

Markers or groups of markers that correlate with the symptoms or occurrence of disease can be used to diagnose disease or predisposition to disease without regard to phenotypic manifestation. To diagnose disease or predisposition to disease, individuals are tested for the presence or absence of polymorphic markers or marker sets that correlate with one or more diseases. If, for example, the presence of allele A1 at polymorphism A correlates with coronary artery disease then individuals with allele A1 at polymorphism A may be at an increased risk for the condition.

Individuals can be tested before symptoms of the disease develop. Infants, for example, can be tested for genetic diseases such as phenylketonuria at birth. Individuals of any age could be tested to determine risk profiles for the occurrence of future disease. Often early diagnosis can lead to more effective treatment and prevention of disease through dietary, behavior or pharmaceutical interventions. Individuals can also be tested to determine carrier status for genetic disorders. Potential parents can use this information to make family planning decisions.

Individuals who develop symptoms of disease that are consistent with more than one diagnosis can be tested to make a more accurate diagnosis. If, for example, symptom S is consistent with diseases X, Y or Z but allele A1 at polymorphism A correlates with disease X but not with diseases Y or Z, an individual with symptom S is tested for the presence or absence of allele A1 at polymorphism A. Presence of allele A1 at polymorphism A is consistent with a diagnosis of disease X. Genetic expression information discovered through the use of arrays has been used to determine the specific type of cancer a particular patient has (Golub et al., 2001, Science 286:531-537, hereby incorporated by reference in its entirety for all purposes.).

The results of the assays described herein are also inherently useful as a guide for treatment or prevention of Aβ deposition in response to TBI.

### Pharmacogenomics

Pharmacogenomics refers to the study of how genes affect response to drugs. There is great heterogeneity in the way individuals respond to medications, in terms of both host toxicity and treatment efficacy. There are many causes of this variability, including: severity of the disease being treated; drug interactions; and the individuals age and nutritional status. Despite the importance of these clinical variables, inherited differences in the form of genetic polymorphisms can have an even greater influence on the efficacy and toxicity of medications. Genetic polymorphisms in drug-metabolizing enzymes, transporters, receptors, and other drug targets have been linked to interindividual differences in the efficacy and toxicity of many medications (Evans and Relling, 2001, Science 286:487-491, which is herein incorporated by reference for all purposes).

An individual patient has an inherited ability to metabolize, eliminate and respond to specific drugs. Correlation of polymorphisms with pharmacogenomic traits identifies those polymorphisms that impact drug toxicity and treatment efficacy. This information can be used by doctors to determine what course of medicine is best for a particular patient and by pharmaceutical companies to develop new drugs that target a particular disease or particular individuals within the population, while decreasing the likelihood of adverse affects. Drugs can be targeted to groups of individuals who carry a specific allele or group of alleles. For example, individuals who carry allele A1 at polymorphism A may respond best to medication X while individuals who carry allele A2 respond best to medication Y. A trait may be the result of a single polymorphism but will often be determined by the interplay of several genes.

In addition some drugs that are highly effective for a large percentage of the population, prove dangerous or even lethal for a very small percentage of the population. These drugs typically are not available to anyone. Pharmacogenomics can be used to correlate a specific genotype with an adverse drug response. If pharmaceutical companies and physicians can accurately identify those patients who would suffer adverse responses to a particular drug, the drug can be made available on a limited basis to those who would benefit from the drug.

Similarly, some medications may be highly effective for only a very small percentage of the population while proving only slightly effective or even ineffective to a large percentage of patients. Pharmacogenomics allows pharmaceutical companies to predict which patients would be the ideal candidate for a particular drug, thereby dramatically reducing failure rates and providing greater incentive to companies to continue to conduct research into those drugs.

### Allele Frequency Determination

Large numbers of individuals, for example, 20, 40, 60, 100, 1000, 10,000, Or 100,000 or more may be genotyped at a particular SNP to determine the frequency of each of the possible alleles. Results from different populations may be compared to determine if some alleles are present at higher or lower frequencies in distinct populations. Some SNPs may be identified that are monomorphic (zero-heterozygosity) in one population but not in another population. Allele frequencies may be used to study phenomenon such as natural selection, random genetic drift, demographic evens such as population bottlenecks or expansions or combinations of these.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods employed in the experiments disclosed herein are now described.

### Cohort Population

A cohort of human traumatic brain injury cases (N=81) was selected from a diagnostic neuropathology archive. These brains were analyzed for the presence or absence of amyloid plaques. The cohort was then divided into an amyloid positive group (n=19) and a control, or amyloid negative (n=62), group for the purposes of this study.

### Genetic Analysis

Tissue from the brains of the study cohort cases was subjected to genetic analysis to determine the length of the GT repeat present in the Neprilysin promoter region.

DNA was extracted from the samples using standard methods well-known in the art. The regions of the Neprilysin gene containing each of the dinucleotide repeats of interest were amplified by polymerase chain reaction (PCR) using the following primers: Forward 5′-TTTCAGTATGAATTCCGCAGT-3′ (SEQ ID No. 3); Reverse 5′-TGATCCCTTTCCTCTTTTGAAT-3′ (SEQ ID No. 4).

Fragment analyses of the amplified GT repeats were performed using the Beckman Coulter CEQ 8000 genetic analysis system. Cases that were identified as amyloid positive were compared to controls with respect to the GT polymorphism allele present. Comparisons between groups were performed statistically using the Fisher's Exact test.

The results of the experiments presented in this Example are now described.

### Example 1

### Identification of Alleles and Genotypes Present in the Study Cohort

Genetic analysis revealed the presence of 5 individual alleles and 10 genotypes within the study cohort. Each allele is defined by the number of GT repeats found for that polymorphism and named accordingly. Individual alleles for 19, 20, 21, 22, and 23 GT repeats within the NEP promoter were all observed in the present study's patient cohort.

The genotypes are described according to the number of GT repeats encode by each allele found to be present in an individual, and are as follows: 19/20; 19/21; 19/22; 20/20; 20/21; 20/22; 20/23; 21/21; 21/22; and 21/23.

### Example 2

### Association of NEP Polymorphisms with Aβ Disposition in Subjects with Traumatic Brain Injury (TBI)

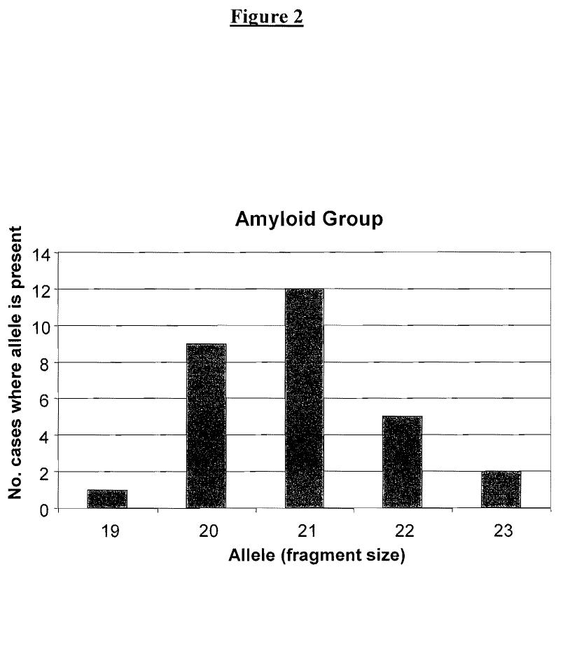

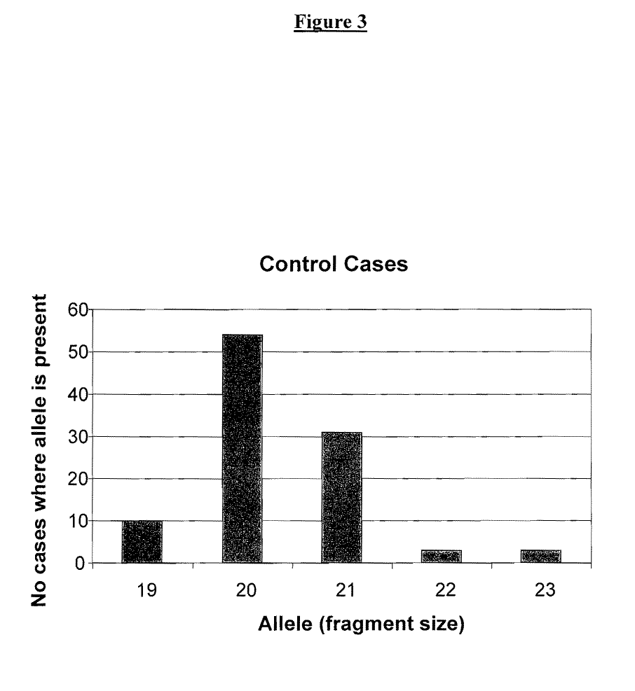

Among the patient cohort with traumatic brain injury (TBI), NEP polymorphisms that encode longer length of the GT repeat were more prevalent in cases with Aβ deposition (FIG. 2) as compared to controls that did not exhibit Aβ deposition (FIG. 3). Table 1 depicts the frequency of the allele encoding exactly 20 GT repeats occurs in control (amyloid negative) and amyloid positive individuals.

Table 2 depicts the frequency of the allele encoding exactly 22 GT repeats occurs in control (amyloid negative) and Aβ positive individuals. These data indicate that presence of allele 22 was independently associated with an increased risk of having Aβ plaques (p<0.003) in patients with TBI, where as the presence of allele 20 (p<0.03) was associated with a decreased risk.

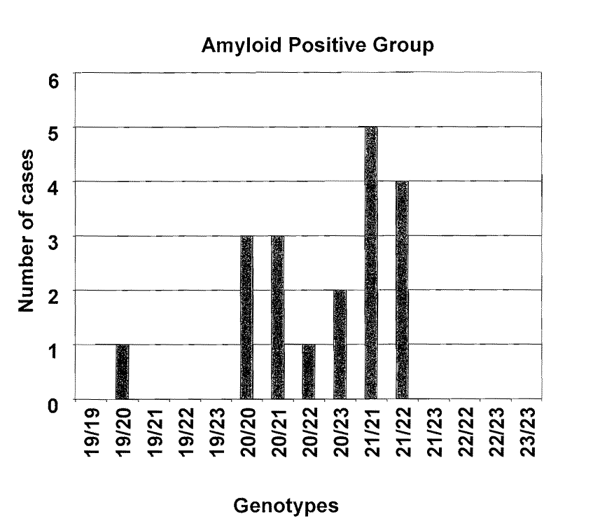

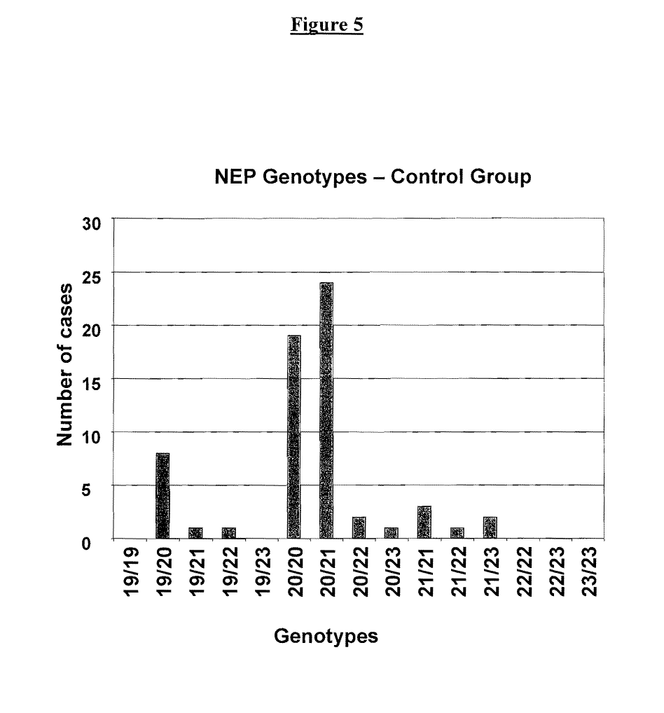

Each member of the study cohort was genotyped according to the GT repeat polymorphism detected using PCR and fragment analysis. FIG. 4 depicts the distribution of NEP polymorphic genotypes among individual with TBI that also were positive for Aβ plaques. FIG. 5 depicts the distribution of NEP polymorphic genotypes among individuals with TBI that also were negative for Aβ plaques (i.e. controls). Although no one genotype was identified as significant, the graph depicting the distribution of genotypes for the Aβ positive subjects is skewed to the right, indicating that this population in general expressed NEP polymorphisms that encoded longer GT repeats in the NEP gene.

A significantly increased risk of Aβ deposition is associated with a genotype greater than a total of 41 repeats (p<0.0001) as demonstrated in Table 3. Thus, by way of a non-limiting example, an individual with an allele encoding 20 GT repeats and another allele encoding 22 GT repeats has a total repeat count of 42 and would be at significantly increased risk of developing Aβ plaques in response to TBI. In contrast, an individual with each allele encoding 19 GT repeats would have a total of 38 GT repeats and would be at decreased risk of developing Aβ plaques in response to TBI.

TABLE 1
Presence of 20 GT Repeats by Amyloid Status

| Presence of Exactly | | | | |
|---|---|---|---|---|
| 20 GT Repeats in At | | Amyloid Status | | |
| Least One Allele | | Negative | Positive | Total |
| No | Frequency | 8 | 9 | 17 |
| | Percent | 9.88 | 11.11 | 20.99 |
| | Row Percent | 47.06 | 52.94 | |
| | Column Percent | 12.9 | 47.37 | |
| Yes | Frequency | 54 | 10 | 64 |
| | Percent | 66.67 | 12.35 | 79.01 |
| | Row Percent | 84.38 | 15.63 | |
| | Column Percent | 87.1 | 52.63 | |
| Total | Frequency | 62 | 19 | 81 |
| | Percent | 76.54 | 23.46 | 100 |

Fisher's Exact Test
P-Value 0.0028

TABLE 2
Presence of 22 GT Repeats by Amyloid Status

| Presence of Exactly | | | | |
|---|---|---|---|---|
| 22 GT Repeats in At | | Amyloid Status | | |
| Least One Allele | | Negative | Positive | Total |
| No | Frequency | 58 | 14 | 72 |
| | Percent | 71.6 | 17.28 | 88.89 |
| | Row Percent | 80.56 | 19.44 | |
| | Column Percent | 93.55 | 73.68 | |
| Yes | Frequency | 4 | 5 | 9 |
| | Percent | 4.94 | 6.17 | 11.11 |
| | Row Percent | 44.44 | 55.56 | |
| | Column Percent | 6.45 | 26.32 | |
| Total | Frequency | 62 | 19 | 81 |
| | Percent | 76.54 | 23.46 | 100 |

Fisher's Exact Test
P-Value 0.0292

All p-values are based on the Fisher's Exact test.

TABLE 3
Greater Than 41 Total GT Repeats by Amyloid Status

| Greater Than 41 | | Amyloid Status | | |
|---|---|---|---|---|
| Total GT Repeats | | Negative | Positive | Total |
| No | Frequency | 53 | 7 | 60 |
| | Percent | 65.43 | 8.64 | 74.07 |
| | Row Percent | 88.33 | 11.67 | |
| | Column Percent | 85.48 | 36.84 | |
| Yes | Frequency | 9 | 12 | 21 |
| | Percent | 11.11 | 14.81 | 25.93 |
| | Row Percent | 42.86 | 57.14 | |
| | Column Percent | 14.52 | 63.16 | |
| Total | Frequency | 62 | 19 | 81 |
| | Percent | 76.54 | 23.46 | 100 |

Fisher's Exact Test
P-Value 8.21E−05

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of detecting a neprilysin gene polymorphism associated with amyloid β plaque development after traumatic brain injury (TBI) in a human, said method comprising obtaining a body sample from said human; isolating genomic DNA from said sample; amplifying the neprilysin gene from said genomic DNA; and detecting the number of GT tandem repeats present in the promoter region of said NEP gene, wherein when said human expresses a NEP polymorphism having two alleles with a total of more than 41 GT repeats in the promoter of the NEP gene, said human is at risk of developing amyloid β plaque after traumatic brain injury.

2. A method of detecting a neprilysin gene polymorphism associated with amyloid β plaque development after traumatic brain injury in a human, said method comprising obtaining a body sample from said human; isolating genomic DNA from said sample; amplifying the neprilysin gene from said genomic DNA; and detecting the number of GT tandem repeats present in the promoter region of said NEP gene, wherein when said human expresses a NEP polymorphism comprising a single allele encoding 22 GT repeats in the promoter of the NEP gene, said human is at risk of developing amyloid β plaque after traumatic brain injury.

3. A method of detecting a neprilysin gene polymorphism associated with amyloid β plaque development after traumatic brain injury in a human, said method comprising obtaining a body sample from said human; isolating genomic DNA from said sample; amplifying the neprilysin gene from said genomic DNA; and detecting the number of GT tandem repeats present in the promoter region of said NEP gene, wherein when said human expresses a NEP polymorphism comprising a single allele encoding 20 GT repeats in the promoter of the NEP gene, said human has a reduced risk of developing amyloid β plaque after traumatic brain injury.

4. A method of evaluating a human's risk of developing an amyloid β plaque after traumatic brain injury, said method comprising obtaining a body sample from said human; isolating genomic DNA from said sample; amplifying the neprilysin gene from said genomic DNA; and detecting the number of GT tandem repeats present in the promoter region of said NEP gene, wherein when said human expresses a NEP polymorphism having two alleles with a total of more than 41 GT repeats in the promoter of the NEP gene, said human is at increased risk of developing amyloid β plaque after traumatic brain injury.

5. A method of evaluating a human's risk of developing an amyloid β plaque after traumatic brain injury, said method comprising obtaining a body sample from said human; isolating genomic DNA from said sample; amplifying the neprilysin gene from said genomic DNA; and detecting the number of GT tandem repeats present in the promoter region of said NEP gene, wherein when said human expresses a NEP polymorphism comprising a single allele encoding 22 GT repeats in the promoter of the NEP gene, said human is at increased risk of developing amyloid β plaque after traumatic brain injury.

6. A method of evaluating a human's risk of developing an amyloid β plaque after traumatic brain injury, said method comprising obtaining a body sample from said human; isolating genomic DNA from said sample; amplifying the neprilysin gene from said genomic DNA; and detecting the number of GT tandem repeats present in the promoter region of said NEP gene, wherein when said human expresses a NEP polymorphism comprising a single allele encoding 20 GT repeats in the promoter of the NEP gene, said human is at a reduced risk of developing amyloid β plaque after traumatic brain injury.


SEQUENCE LISTING (4 sequences)

SEQ ID NO: 1 - DNA, 5643 bp, Homo sapiens
gagagcgccgagacgcgcggggcgcggagatgtgcaagtggcgaagcttgaccgagagcaggctggagcagccgcccaactcctggcgcgggatctgctgaggggtcacgattttaggtgatgggcaagtcagaaagtcagatggatataactgatatcaacactccaaagccaaagaagaaacagcgatggactccactggagatcagcctctcggtccttgtcctgctcctcaccatcatagctgtgacaatgatcgcactctatgcaacctacgatgatggtatttgcaagtcatcagactgcataaaatcagctgctcgactgatccaaaacatggatgccaccactgagccttgtacagactttttcaaatatgcttgcggaggctggttgaaacgtaatgtcattcccgagaccagctcccgttacggcaactttgacattttaagagatgaactagaagtcgttttgaaagatgtccttcaagaacccaaaactgaagatatagtagcagtgcagaaagcaaaagcattgtacaggtcttgtataaatgaatctgctattgatagcagaggtggagaacctctactcaaactgttaccagacatatatgggtggccagtagcaacagaaaactgggagcaaaaatatggtgcttcttggacagctgaaaaagctattgcacaactgaattctaaatatgggaaaaaagtccttattaatttgtttgttggcactgatgataagaattctgtgaatcatgtaattcatattgaccaacctcgacttggcctcccttctagagattactatgaatgcactggaatctataaagaggcttgtacagcatatgtggattttatgatttctgtggccagattgattcgtcaggaagaaagattgcccatcgatgaaaaccagcttgctttggaaatgaataaagttatggaattggaaaaagaaattgccaatgctacggctaaacctgaagatcgaaatgatccaatgcttctgtataacaagatgacattggcccagatccaaaataacttttcactagagatcaatgggaagccattcagctggttgaatttcacaaatgaaatcatgtcaactgtgaatattagtattacaaatgaggaagatgtggttgtttatgctccagaatatttaaccaaacttaagcccattcttaccaaatattctgccagagatcttcaaaatttaatgtcctggagattcataatggatcttgtaagcagcctcagccgaacctacaaggagtccagaaatgctttccgcaaggccctttatggtacaacctcagaaacagcaacttggagacgttgtgcaaactatgtcaatgggaatatggaaaatgctgtggggaggctttatgtggaagcagcatttgctggagagagtaaacatgtggtcgaggatttgattgcacagatccgagaagtttttattcagactttagatgacctcacttggatggatgccgagacaaaaaagagagctgaagaaaaggccttagcaattaaagaaaggatcggctatcctgatgacattgtttcaaatgataacaaactgaataatgagtacctcgagttgaactacaaagaagatgaatacttcgagaacataattcaaaatttgaaattcagccaaagtaaacaactgaagaagctccgagaaaaggtggacaaagatgagtggataagtggagcagctgtagtcaatgcattttactcttcaggaagaaatcagatagtcttcccagccggcattctgcagccccccttctttagtgcccagcagtccaactcattgaactatgggggcatcggcatggtcataggacacgaaatcacccatggcttcgatgacaatggcagaaactttaacaaagatggagacctcgttgactggtggactcaacagtctgcaagtaactttaaggagcaatcccagtgcatggtgtatcagtatggaaacttttcctgggacctggcaggtggacagcaccttaatggaattaatacactgggagaaaacattgctgataatggaggtcttggtcaagcatacagagcctatcagaattatattaaaaagaatggcgaagaaaaattacttcctggacttgacctaaatcacaaacaactatttttcttgaactttgcacaggtgtggtgtggaacctataggccagagtatgcggttaactccattaaaacagatgtgcacagtccaggcaatttcaggattattgggactttgcagaactctgcagagttttcagaagcctttcactgccgcaagaattcatacatgaatccagaaaagaagtgccgggtttggtgatcttcaaaagaagcattgcagcccttggctagacttgccaacaccacagaaatggggaattctctaatcgaaagaaaatgggccctaggggtcactgtactgacttgagggtgattaacagagagggcaccatcacaatacagataacattaggttgtcctagaaagggtgtggagggaggaagggggtctaaggtctatcaagtcaatcatttctcactgtgtacataatgcttaatttctaaagataatattactgtttatttctgtttctcatatggtctaccagtttgctgatgtccctagaaaacaatgcaaaacctttgaggtagaccaggatttctaatcaaaagggaaaagaagatgttgaagaatacagttaggcaccagaagaacagtaggtgacactatagtttaaaacacattgcctaactactagtttttacttttatttgcaacatttacagtccttcaaaatccttccaaagaattcttatacacattggggccttggagcttacatagttttaaactcatttttgccatacatcagttattcattctgtgatcatttattttaagcactcttaaagcaaaaaatgaatgtctaaaattgttttttgttgtacctgctttgactgatgctgagattcttcaggcttcctgcaattttctaagcaatttcttgctctatctctcaaaacttggtatttttcagagatttatataaatgtaaaaataataatttttatatttaattattaactacatttatgagtaactattattataggtaatcaatgaatattgaagtttcagcttaaaataaacagttgtgaaccaagatctataaagcgatatacagatgaaaatttgagactatttaaacttataaatcatattgatgaaaagatttaagcacaaactttagggtaaaaattgccattggacagttgtctagagatatatatacttgtggttttcaaattggactttcaaaattaaatctgtccctgagagtgtctctgataaaagggcaaatctgcacctatgtagctctgcatctcctgtcttttcaggtttgtcatcagatggaaatattttgataataaattgaaattgtgaactcattgctccctaagactgtgacaactgtctaactttagaagtgcatttctgaatagaaatgggaggcctctgatggaccttctagaattataagtcacaaagagttctggaaaagaactgtttactgcttgataggaattcatcttttgaggcttctgttcctctcttttcctgttgtattgactattttcgttcattacttgattaagattttacaaaagaggagcacttccaaaattcttatttttcctaacaaaagatgaaagcagggaatttctatctaaatgatgagtattagttccctgtctcttgaaaaatgcccatttgcctttaaaaaaaaaagttacagaaatactataacatatgtacataaattgcataaagcataagtatacagttcaataaacttaactttaactgaacaatggccctgtagccagcacctgtaagaaacagagcagtaccagcgctctaaaagcacctccttgtcactttattactcccagaacaacaactatcctgacttctaatatcattcactagctttgcctggttttgtcttttatgcagatagaatcaatcagtatgtattcttttgtgcctggcttctttctctcagccttacatttgtgagattcctctgtattgtgctgattgtggatcttttcattctcattgcagaataatgttctattgtgggacttattacaatttgttcatcctattgttgatgggcacttgagaactttccattttggcgctattacaaatagtgcaactatgaatgtactgcatgttaccatcttacttgagcctttaatggacttatttcttcaaatccttccaaaaattattataagcattgaaattatagtttcaagccaactgtggatacccttaccctttcctcctttatcacaaccaccgttacaagtatacttatatttccctaaaatacatttaaaacttacctaagtgacatttgtagttggagtaataggagcttccagctctaataaaacagctgtctctaacttattttatttccatcatgtcagagcaggtgaagagccagaagtgaagagtgactagtacaaattataaaaagccactagactcttcactgttagctttttaaaacattaggctcccatccctatggaggaacaactctccagtgcctggatcccctctgtctacaaatataagattttctgggcctaaaggatagatcaaagtcaaaaatagcaatgcctccctatccctcacacatccagacatcatgaattttacatggtactcttgttgagttctgtagagccttctgatgtctctaaagcactaccgattctttggagttgtcacatcagataagacatatctctaattccatccataaatccagttctactatggctgagttctggtcaaagaaagaaagtttagaagctgagacacaaagggttgggagctgatgaaactcacaaatgatggtaggaagaagctctcgacaatacccgttggcaaggagtctgcctccatgctgcagtgttcgagtggattgtaggtgcaagatggaaaggattgtaggtgcaagctgtccagagaaaagagtccttgttccagccctattctgccactcctgacagggtgaccttgggtatttgcaatattcctttgggcctctgcttctctcacctaaaaaaagagaattagattatattggtggttctcagcaagagaaggagtatgtgtccaatgctgccttcccatgaatctgtctcccagttatgaatcagtgggcaggataaactgaaaactcccatttacgtgtctgaatcgagtgagacaaaattttagtccaaataacaagtaccaaagttttatcaagtttgggtctgtgctgctgttactgttaaccatttaagtggggcaaaaccttgctaattttctcaaaagcatttatcattcttgttgccacagctggagctctcaaactaaaagacatttgttattttggaaagaagaaagactctattctcaaagtttcctaatcagaaatttttatcagtttccagtctcaaaaatacaaaataaaaacaaacgtttttaatactattgcttttatgcctagtcaactctg

SEQ ID NO: 2 - protein, 750 aa, Homo sapiens
MGKSESQMDITDINTPKPKKKQRWTPLEISLSVLVLLLTIIAVTMIALYATYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 3 - DNA, 21 bp, artificial
  Chemically synthesized
tttcagtatgaattccgcagt

SEQ ID NO: 4 - DNA, 22 bp, artificial
  Chemically synthesized
tgatccctttcctcttttgaat